(19) United States
(12) Patent Application Publication
(10) Pub. No.: US 2016/0206592 A1
(43) Pub. Date: Jul. 21, 2016

(54) METHOD FOR TREATING AMYOTROPHIC LATERAL SCLEROSIS BY INHIBITION OF CXCR4/CXCL12 SIGNALING

(71) Applicant: RAMOT AT TEL-AVIV UNIVERSITY LTD., Tel Aviv (IL)

(72) Inventors: Beka SOLOMON, Herzlia Pituach (IL); Inna RABINOVICH-NIKITIN, Ashdod (IL); Beka BARBIRO, Rishon-LeZion (IL)

(73) Assignee: RAMOT AT TEL-AVIV UNIVERSITY LTD., Tel-Aviv (IL)

(21) Appl. No.: 14/915,242

(22) PCT Filed: Aug. 29, 2014
(86) PCT No.: PCT/US14/53352

§ 371 Date: Feb. 28, 2016

Related U.S. Application Data
(60) Provisional application No. 61/871,954, filed on Aug. 30, 2013.

(51) Int. Cl. A61K 31/395

(52) U.S. Cl. CPC A61K 31/395

(57) ABSTRACT

The present invention provides use of an inhibitor of G protein-coupled receptor CXCR4 for treating amyotrophic lateral sclerosis (ALS), for inhibiting glutamate release in astrocytes, or for increasing remyelinization in motor neurons.

### BACKGROUND OF THE INVENTION

1. Field of the Invention

This invention generally relates to the fields of amyotrophic lateral sclerosis (ALS) pathology and CXCR4/CXCL12 signaling.

2. Description of the Related Art

Amyotrophic lateral sclerosis (ALS) is the most common and most aggressive form of adult motor neuron (MN) degeneration. The cause of the disease is still unknown, but some protein mutations have been linked to the pathological process. The pathophysiology of neurodegenerative diseases is complex which includes cholinergic deficit, glutamate excitatoxicity, neuroinflammation, immunity dysregulation, glucose hypometabolism and blood-central nervous system barrier (B-CNS-B) disruption.

Astrocytic cells are considered to have a primary role in the pathological process of amyotrophic lateral sclerosis (ALS), and are substantial contributers to motor neuron death. Astroglial abnormalities, such as changes in the release and uptake of astrocytic glutamate preface clinical symptoms of the disease (Vargas et al., 2010).

Chemokines and their receptors in the central nervous system (CNS) are relevant for the understanding of brain physiology and pathophysiology and may lead to the development of targeted treatments for neurodegenerative diseases. Chemokines are known to be plurifunctional and active on many different cell types, including neurons and glial cells.

Chemokine receptors, including the G-protein-coupled receptor CXCR4, are expressed widely in neurons and glial cell. The ligand of CXCR4, the chemokine stromal-derived factor 1 (SDF-1), also known as CXCL12, evokes glutamate release and thereby modulates neuronal function or apoptosis. The mechanism of action starts with binding of CXCL12 to CXCR4, increase in [Ca2+], stimulation of extracellular signal related kinases and release of TNFα from astrocyte and microglia cell surface (Allen et al., 2001).

AMD3100 (1,1′-[1,4-Phenylenebis(methylene)]bis-1,4,8,11-tetraazacyclotetradecane) is a bicyclam molecule that specifically and reversibly blocks SDF-1 binding to CXCR4. AMD3100 has been shown to rapidly mobilize hematopoietic stem and progenitor cells (HSPCs) from the bone marrow (BM) into the blood of mice, non-human primates and humans. Disruption of CXCR4 signaling by AMD3100 was seen to inhibit the migration activity of grafted neuronal stem/progenitor cells, as observed in hemiplegic mice (Arimitsu et al., 2012). In 2008, AMD3100 was FDA-approved for HSPC mobilization in combination with granulocyte colony stimulating factor (G-CSF) in patients with non-Hodgkin's lymphoma and multiple myeloma undergoing autologous transplantation (Pusic et al., 2010).

Other substantial clinical feature of AMD3100 is promoting mobilization of CXCR4⁺VEGFR1⁺ cells through modulation of plasma SDF-1 levels, suggesting that AMD3100 has a regulatory role in the recruitment of pro-angiogenic cells and in the extent of revascularization (Petit et al., 2007), which is extremely important in maintenance and function of central nervous system (CNS) neurons.

In ALS patients and rodents expressing ALS-associated superoxide dismutase 1 (SOD1) mutations alterations were reported in the blood-Central Nervous System barrier (B-CNS-B) composed of the blood brain barrier (BBB), blood-spinal cord barrier (BSCB), and blood-cerebrospinal fluid barrier (BCSFB) as suggested from the reduction of levels of various tight junction proteins including ZO-1, occludin and claudin-5 between endothelial cells. The loss of tight junction proteins occludin and ZO-1 in the microvasculature has been also shown to be mediated by various cytokines such as monocyte chemoattractant protein-1 (MCP1), TNF-α, IL-1β, and IFN-γ. The reduction of the tight junction proteins resulted in microhemorrhage with release of neurotoxic hemoglobin-derived products, reductions in microcirculation and hypoperfusion. SOD1 mutants are proposed to mediate endothelial damage even before motor neuron death and hypoxia and inflammation led to increased BSCB permeability and disruption. Early motor-neuron dysfunction and injury were shown to be proportional to the degree of BSCB disruption, and early protection of the BSCB integrity was found to delay onset of motor-neuron impairment and degeneration. Altogether, these findings in mice show that BSCB breakdown plays a role in early-stage disease pathogenesis and that restoring BSCB integrity retards the disease process.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention provides a method for treating amyotrophic lateral sclerosis (ALS) which involves administering to a patient suffering from ALS an effective amount of an inhibitor of G protein-coupled receptor CXCR4.

In addition to use of an inhibitor of G protein-coupled receptor CXCR4 for treating amyotrophic lateral sclerosis (ALS), the present invention further provides for the use of an inhibitor of G protein-coupled receptor CXCR4 for inhibiting glutamate release in astrocytes or for increasing remyelinization in motor neurons.

### BRIEF DESCRIPTION OF THE DRAWINGS

FIG. 1 is a Western blot analysis of CXCR4 and tubulin in transgenic G93A mice and non-transgenic litter mates (LM) at 30, 80 and 100 days old and at the final stage of the disease.

FIG. 2 is a graph showing CXCR4 levels in transgenic G93A mice and non-transgenic litter mates throughout disease progression (30, 80 and 100 days old and at the final stage) as a ratio of CXCR4 to actin.

FIGS. 3A-3B are graphs showing survival (FIG. 3A), weight change (FIG. 3B), and performance on the Rotarod test (FIG. 3C) for G93A female mice subcutaneously treated with either AMD3100 or PBS, starting at 70 days old. Six mice received AMD3100 once a week, eight mice received AMD3100 twice a week, and seven mice received PBS once a week.

FIGS. 4A-4C are graphs showing survival (Kaplan-Meier curves; P-value=0.0001) of SOD1-G93A female and male mice subcutaneously treated with either 5 mg/kg AMD3100 or PBS, starting at 50 days old; N=29 (AMD3100), N=16 (PBS). The survival for males and females together was 12 days according to Kaplan-Meier curve (mean AMD3100=144; mean PBS=131; median AMD3100=143; median PBS=131) (FIG. 4A). The survival for females only injected twice a week with 5 mg/kg AMD3100 was 14 days (mean AMD3100=146; mean PBS=130; median AMD3100=144; median PBS=130) with N=17 (AMD3100) and N=9 (PBS) (FIG. 4B) The survival for males only injected twice a week with 5 mg/kg AMD3100 was 8 days (mean AMD3100=141; mean PBS=133; median AMD3100=139; median PBS=131) with N=12 (AMD3100) and N=7 (PBS) (FIG. 4C).

FIGS. 5A-5B show the effect of AMD3100 treatment on body weight and motor function of female SOD1-G93A mice. FIG. 5A shows 50 days old female SODG93A mice that were s.c. injected with AMD3100 or PBS. Weight of each animal was recorded weekly. N (PBS)=9, N(AMD3100)=17.) FIG. 5B shows motor function of the female mice which was assessed by performing Rotorod test. The animals were trained to run on the 2-cm-diameter rod, which rotated at accelerating speed from 10 turns per minute to 40 turns per minute. The mice were allowed to run for up to 5 min in each trial, or until they fell off.

FIG. 6 shows increase in survival of NSC-34 cells stably transfected with mutant SOD1 which incubated with primary astrocytes treated with AMD3100. Primary astrocytes from 3 days old newborn SODG93A mice were incubated AMD3100 for 24 h. Media of treated astrocytes was then added to NSC-34 cells stably transfected with mutant SOD1. 25 ug AMD3100 increased the survival of motor neuron like cells, compared to other concentrations.

FIGS. 7A-7B show inflammation levels displayed by activated microglia levels, following AMD3100 treatment. 50 days old female SODG93A mice were s.c injected with 5 mg/kg AMD3100 twice a week. On 110 days old mice were sacrificed and analyzed using western blot for inflammatory markers of activated microglia. FIG. 7A. is cd36 levels. FIG. 7B. is Iba-1 levels.

FIGS. 8A-8B show inflammatory cytokines levels following AMD3100 treatment. 50 days old female SODG93A mice were s.c injected with 5 mg/kg AMD3100 twice a week. On 110 days old mice were sacrificed and analyzed for inflammatory cytokines. FIG. 8A: IL-6 levels using western blot analysis. FIG. 8B: TNF-α levels using ELISA.

FIGS. 9A-9C show Blood-CNS-Barrier markers levels following AMD3100 treatment. 50 days old female SODG93A mice were s.c injected with 5 mg/kg AMD3100 twice a week. On 110 days old mice were sacrificed and analyzed for B-CNS-B markers using western blot. FIG. 9A shows ZO-1 levels. FIG. 9B shows claudin 5 levels. FIG. 9C shows Occludin levels.

FIG. 10 shows myelin levels, which is a dielectric material which forms a layer around neuronal axons, essential for increasing the speed at which impulses propagate along the axons and known to be depleted in ALS, following AMD3100 treatment. 50 days old female SODG93A mice were s.c injected with 5 mg/kg AMD3100 twice a week. On 110 days old mice were sacrificed and analyzed for myelin levels using western blot. Myelin levels were significantly increased following AMD3100 treatment.

FIG. 11 shows EAAT2 levels, which is the main transporter of glutamate uptake, known to be proteolytically cleaved by 95% in ALS pathology, following AMD3100 treatment. 50 days old female SODG93A mice were s.c injected with 5 mg/kg AMD3100 twice a week. On 110 days old mice were sacrificed and analyzed for EAAT2 levels using western blot. EAAT2 levels were increased following AMD3100 treatment.

### DETAILED DESCRIPTION OF THE INVENTION

Inhibition of the CXCR4/CXCL12 signaling via an antagonist of CXCR4 receptor, AMD3100, in an ALS mouse model was shown by the present inventors to result in beneficial effect for amyotrophic lateral sclerosis (ALS) such as improvement of motor function, weight loss and extension of life span.

The present invention thus provides a method for treating amyotrophic lateral sclerosis (ALS) and involves administering to a patient suffering from ALS an effective amount of an antagonist/inhibitor of G protein-coupled receptor CXCR4 (use thereof in treating ALS). The present invention is also directed to use of the antagonist/inhibitor in inhibiting CXCR4/CXCL12 signaling and inhibiting the toxic cascade of glutamate release from astrocytes and the eventual neuronal degeneration in ALS. The present invention is further directed to the antagonist/inhibitor in increasing remyelinization in motor neurons. Accordingly, also provided by the present invention are a method for inhibiting glutamate release in astrocytes and a method for increasing remyelinization in motor neurons.

The term “treating” with respect to amyotrophic lateral sclerosis is intended to mean substantially inhibiting, slowing or reversing the progression of amyotrophic lateral sclerosis, such as reducing or inhibiting motor neuron (MN) death, or substantially ameliorating one or more clinical symptoms of amyotrophic lateral sclerosis, such as diminished motor function.

The antagonist/inhibitor of CXCR4 is preferably the bicyclam, 1,1′-[1,4-Phenylenebis(methylene)]bis-1,4,8,11-tetraazacyclotetradecane (also known as AMD3100 or Plerixafor) or a pharmaceutically acceptable salt thereof. Antagonists/inhibitors of CXCR4 are well known in the art, and non-limiting examples of such antagonists/inhibitors of CXCR4 include other bicyclams AMD3196, AMD3207, AMD3203, AMD3109, AMD3128, AMD3166, AMD2763, AMD3070, AMD3209, AMD3068, AMD3208, AMD3120, AMD6037, AMD3390, and AMD6038 (Esté et al., 1999).

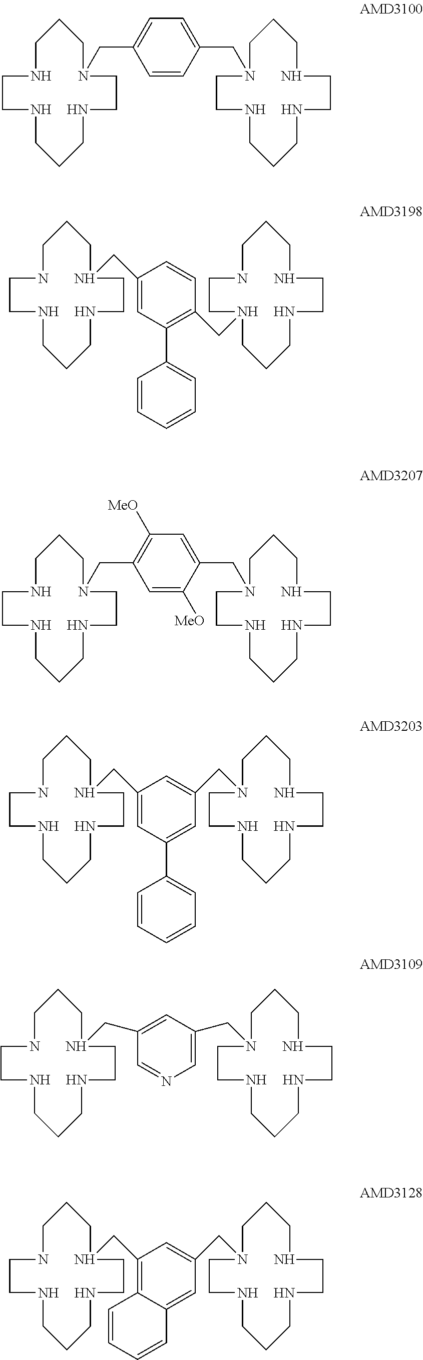 [SMILES: c1cc(CN2CCCNCCNCCCNCC2)ccc1CN1CCCNCCNCCCNCC1 ; c1ccc(-c2cc(CN3CCCNCCNCCCNCC3)ccc2CN2CCCNCCNCCCNCC2)cc1 ; COc1cc(CN2CCCNCCNCCCNCC2)c(OC)cc1CN1CCCNCCNCCCNCC1 ; c1ccc(-c2cc(CN3CCCNCCNCCCNCC3)cc(CN3CCCNCCNCCCNCC3)c2)cc1 ; c1ncc(CN2CCCNCCNCCCNCC2)cc1CN1CCCNCCNCCCNCC1 ; c1ccc2c(CN3CCCNCCNCCCNCC3)cc(CN3CCCNCCNCCCNCC3)cc2c1]

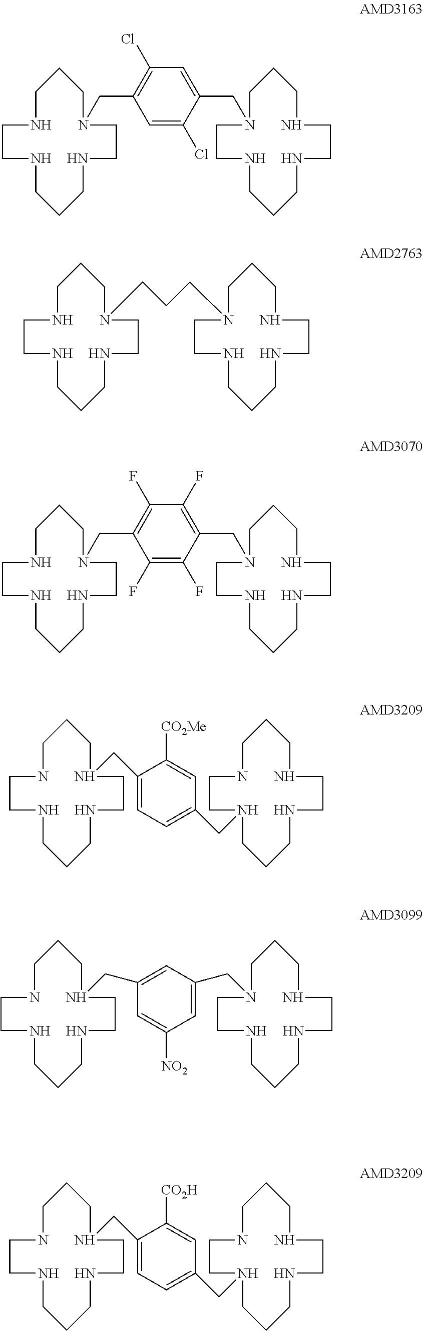 [SMILES: Clc1cc(CN2CCCNCCNCCCNCC2)c(Cl)cc1CN1CCCNCCNCCCNCC1 ; C1CNCCNCCCN(CCCN2CCCNCCNCCCNCC2)CCNC1 ; Fc1c(F)c(CN2CCCNCCNCCCNCC2)c(F)c(F)c1CN1CCCNCCNCCCNCC1 ; COC(=O)c1cc(CN2CCCNCCNCCCNCC2)ccc1CN1CCCNCCNCCCNCC1 ; O=[N+]([O-])c1cc(CN2CCCNCCNCCCNCC2)cc(CN2CCCNCCNCCCNCC2)c1 ; O=C(O)c1cc(CN2CCCNCCNCCCNCC2)ccc1CN1CCCNCCNCCCNCC1]

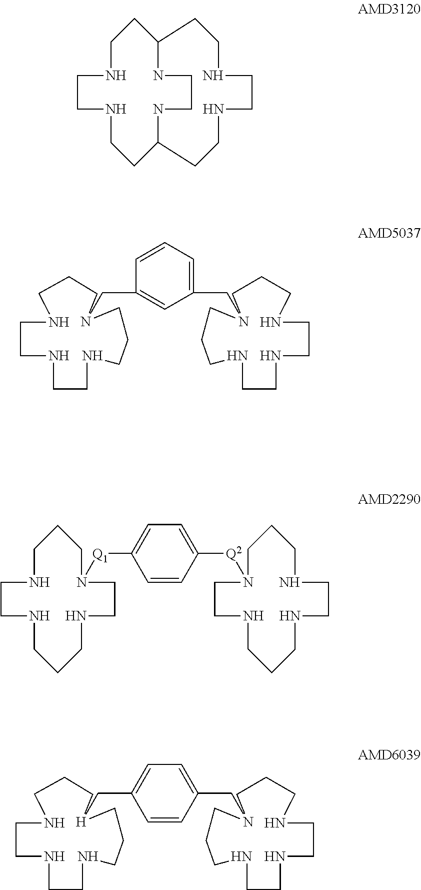

Transition metal ions, preferably Cu2+, Zn2+, or Ni2+, may be incorporated into the cyclam rings of the above compounds (Gerlach et al., 2003).

Further non-limiting examples of antagonists/inhibitors of CXCR4 for use in the present invention include the CXCR4 peptide antagonist CTCE-9908 (amino acid sequence KGVSLSYR-X-RYSLSVGK; Faber et al., 2007) and those disclosed in U.S. Pat. Nos. 8,519,124; 7,435,718; 7,423,011; 8,476,290; 8,153,625; 7,964,191; 7,932,281; and 5,583,131 (and RE42,152), which are incorporated herein by reference.

As used herein, a “pharmaceutical composition” refers to a pyrogen-free preparation of an effective amount of a CXCR4 antagonist/inhibitor, as the active ingredient, with other chemical components such as physiologically suitable carriers and excipients. The purpose of a pharmaceutical composition is to facilitate administration of a compound to a patient.

The phrases “physiologically acceptable carrier” and “pharmaceutically acceptable carrier,” which may be interchangeably used, refer to a carrier or a diluent that does not cause significant irritation to the patient and does not abrogate the biological activity and properties of the administered active ingredient.

The term “excipient” refers to an inert substance added to a pharmaceutical composition to further facilitate administration of an active ingredient. Non-limiting examples of excipients include calcium carbonate, calcium phosphate, various sugars and types of starch, cellulose derivatives, gelatin, vegetable oils and polyethylene glycols.

Pharmaceutical formulations may be adapted for administration by any appropriate route, for example by the oral (including buccal or sublingual), rectal, nasal, topical (including buccal, sublingual, or transdermal), vaginal, or parenteral (including subcutaneous, intracutaneous, intramuscular, intra-articular, intrasynovial, intrasternal, intrathecal, intralesional, intravenous, or intradermal injections or infusions) route. Such formulations may be prepared by any method known in the art of pharmacy, for example by bringing into association the active ingredient with the carrier(s) or excipient(s). Techniques for formulation and administration of drugs may be found in “Remington's Pharmaceutical Sciences,” Mack Publishing Co., Easton, Pa., latest edition, which is incorporated herein by reference and are well known in the art.

Suitable routes of administration may, for example, include transnasal or parenteral delivery, including intramuscular, subcutaneous and intramedullary injections as well as administration directly to the brain, e.g., intracranial administration, as well as injection or infusion into the cerebrospinal fluid. In one aspect of this embodiment, administration is by intrathecal injection or infusion, intraventricular injection or infusion, intraparenchymal injection or infusion, or intracerebroventricular injection or infusion. In more specific aspects, administration is by intraparenchymal injection; intracerebroventricular injection; or intracerebroventricular infusion.

Methods for achieving the delivery of the active ingredient and a pharmaceutical composition containing the active ingredient are well known to those skilled in the art of drug delivery. Specific examples include delivery intrathecally by mini-osmotic pump (Ignacio et al., 2005); delivery directly into muscle(s) by syringe or mini osmotic pump (Azzouz et al., 2005); directly administered to peritoneum by syringe or mini osmotic pump (Kieran et al., 2004); delivery directly below the skin by syringe (Reinholz et al., 1999); and delivery directly to the ventricles in the brain by injection or using a small catheter attached to an osmotic pump (Sathasivam et al., 2005). An implant (e.g., small silicon implant) can be placed at muscles or directly onto the spinal cord (Kieran and Greensmith, 2004).

Pharmaceutical compositions for use in accordance with the present invention may be formulated in conventional manner using one or more physiologically acceptable carriers comprising excipients and auxiliaries, which facilitate processing of the active ingredients into preparations which can be used pharmaceutically. Proper formulation is dependent upon the route of administration chosen.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil emulsions.

For instance, for oral administration in the form of a tablet or capsule, the active ingredient component can be combined with an oral, non-toxic pharmaceutically acceptable inert carrier such as ethanol, glycerol, water, and the like. Powders are prepared by comminuting the compound to a suitable fine size and mixing with a similarly comminuted pharmaceutical carrier such as an edible carbohydrate, as, for example, starch or mannitol. Flavoring, preservative, dispersing, and coloring agent can also be present.

Capsules are made by preparing a powder mixture, as described above, and filling formed gelatin sheaths. Glidants and lubricants such as colloidal silica, talc, magnesium stearate, calcium stearate, or solid polyethylene glycol can be added to the powder mixture before the filling operation. A disintegrating or solubilizing agent such as agar-agar, calcium carbonate, or sodium carbonate can also be added to improve the availability of the medicament when the capsule is ingested.

Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, and the like. Lubricants used in these dosage forms include sodium oleate, sodium chloride, and the like. Disintegrators include, without limitation, starch, methyl cellulose, agar, betonite, xanthan gum, and the like. Tablets are formulated, for example, by preparing a powder mixture, granulating or slugging, adding a lubricant and disintegrant, and pressing into tablets. A powder mixture is prepared by mixing the compound, suitable comminuted, with a diluent or base as described above, and optionally, with a binder such as carboxymethylcellulose, an aliginate, gelating, or polyvinyl pyrrolidone, a solution retardant such as paraffin, a resorption accelerator such as a quaternary salt and/or and absorption agent such as betonite, kaolin, or dicalcium phosphate. The powder mixture can be granulated by wetting with a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compounds of the present disclosure can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solution, syrups, and elixirs can be prepared in dosage unit form so that a given quantity contains a predetermined amount of the compound. Syrups can be prepared by dissolving the compound in a suitably flavored aqueous solution, while elixirs are prepared through the use of a non-toxic vehicle. Solubilizers and emulsifiers such as ethoxylated isostearyl alcohols and polyoxyethylene sorbitol ethers, preservatives, flavor additive such as peppermint oil or natural sweeteners, or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax, or the like.

The active ingredient, and pharmaceutically acceptable salts thereof, can also be administered in the form of liposome delivery systems, such as small unilamellar vesicles, large unilamellar vesicles, and multilamellar vesicles. Liposomes can be formed from a variety of phopholipids, such as cholesterol, stearylamine, or phophatidylcholines.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

For injection, the active ingredient may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. A nasal spray, which does not require a pressurized pack or nebulizer as in an inhalation spray, can alternatively used for intranasal administration. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration or administration directly to the brain, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration or administration directly to the brain include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

Pharmaceutical compositions suitable for use in the context of the method of the present invention include compositions wherein the active ingredient is contained in an amount effective to achieve the intended purpose. More specifically, an effective amount means an amount of active ingredient(s) effective to treat ALS, such as, for example, an amount which results in a clinically significant reduction or prevention of neuronal injury or death, or axonal degeneration.

Determination of a therapeutically effective amount is well within the capability of those skilled in the art, especially in light of the detailed disclosure provided herein.

Dosage amount and interval may be adjusted individually to levels of the active ingredient (CXCR4 antagonist/inhibitor) which are sufficient to treat the particular neurodegenerative tauopathy (minimal effective concentration, MEC). Dosages necessary to achieve the MEC will depend on individual characteristics.

Dosage intervals can also be determined using the MEC value. Preparations should be administered using a regimen, which maintains brain levels above the MEC for 10-90% of the time, preferably between 30-90% of the time and most preferably between 50-90% of the time during the course of treatment. The number of administrations will vary depending upon the type and severity of the disease to be treated. In some embodiments, adminstration will be once a month at least until improvement of the condition is achieved. In other embodiments, adminstration will be once every two months, once every three months, once every four months, once every six months or once per year.

Depending on the severity and responsiveness of ALS to be treated in the patient, dosing can be of a single or a plurality of administrations, with the course of treatment lasting from several days to several weeks or until diminution of the disease state is achieved.

The amount of a composition to be administered will, of course, be dependent on the subject being treated, the severity of the affliction, the judgment of the prescribing physician, etc.

Compositions used in the method of the present invention may, if desired, be presented in a pack or dispenser device, such as an FDA approved kit, which may contain one or more unit dosage forms containing the active ingredient. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Having now generally described the invention, the same will be more readily understood through reference to the following example which is provided by way of illustration and is not intended to be limiting of the present invention.

### EXAMPLE

The present inventors predict that inhibition of the CXCR4/CXCL12 signaling by AMD3100 may affect the toxic cascade of glutamate release from astrocytes and the eventual neuronal apoptosis in ALS. To test this hypothesis, experiments were conducted to evaluate the intrinsic CXCR4 receptor levels of a transgenic mice model of ALS and to investigate the changes in CXCR4 levels during disease progression by administering AMD3100, which is known as antagonist for CXCR4 receptor, in order to examine its effect on ALS pathology.

The CXCR4/CXCL12 signaling pathway was inhibited in transgenic G93A mice subcutaneously administered with AMD3100 once and twice a week. The effect of AMD3100 on ALS pathology was examined for improvement in survival, weight loss and motor function and resulted in several pathological beneficial effects.

### Evaluation of CXCR4 Levels Throughout Disease Progression

The transgenic mouse model of ALS expresses high copy number of mutant super oxide dismutase 1 (SOD1) which is a gene that is responsible for up to twenty percent of familial cases of ALS. B6SJL-Tg (SOD1^{G93A}) 1Gur-J mice are the most aggressive murine model of ALS but in many aspects this model mimics clinical symptoms and pathological processes that occur in ALS patients. Spinal cord homogenates of diseased G93A and their non-transgenic litter mates (LM) mice collected throughout disease progression at presymptomatic, symptomatic and final stage were subjected to Western blot analysis in order to determine the CXCR4 levels.

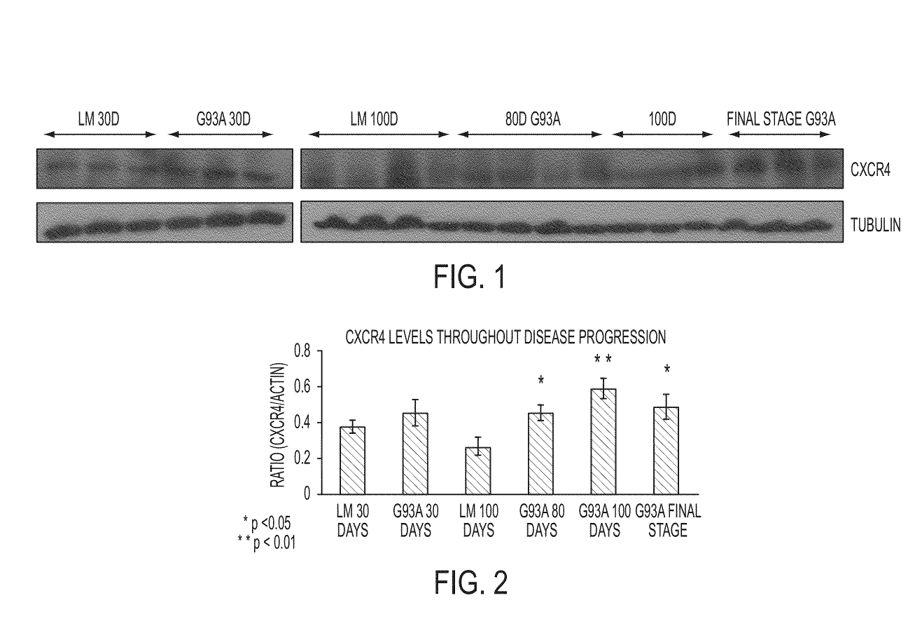

The results in FIGS. 1 and 2 show significant increase in CXCR4 levels of diseased mice compared with their LM throughout disease progression, especially at 100 days old, the onset of the disease. Thus, the evaluated levels of CXCR4 receptor, which is known to bind SDF-1 ligand, showed significant increase as pathology proceeds. While the levels of CXCR4 receptor of 100 days old litter mate (LM) mice are decreased compared with 30 days old LM, the levels of 100 days old and final stage G93A mice are increased, compared with their compatible LM and with 30 days old G93A mice.

### G93A Mice Treated with AMD3100 Once and Twice a Week

According the results of the CXCR4 level evaluation, it may be assumed that since CXCR4 expression is increased throughout disease progression, more stromal-derived factor 1α (SDF1α) binds the receptor (CXCR4), resulting in evoked glutamate release and neuronal apoptosis. Subsequently, by inhibiting CXCR4 with the CXCR4 antagonist AMD3100, the present inventors predict that this would inhibit this signaling pathway.

For this purpose, the present inventors treated G93A female mice subcutaneously with either AMD3100 or PBS, starting at 70 days old. Six mice received AMD3100 once a week, eight mice received AMD3100 twice a week, and seven mice received PBS once a week.

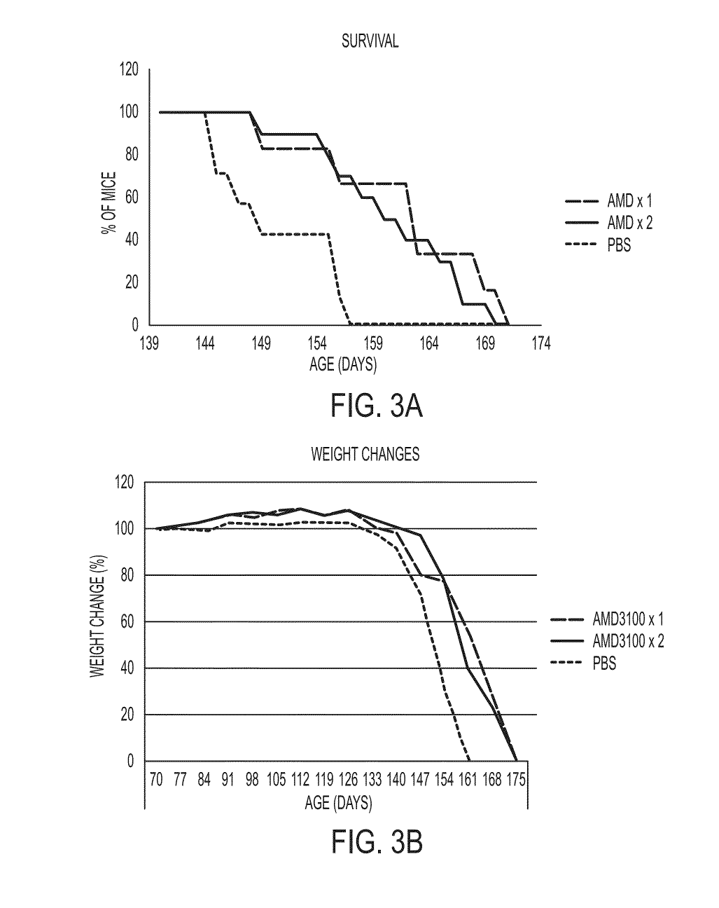

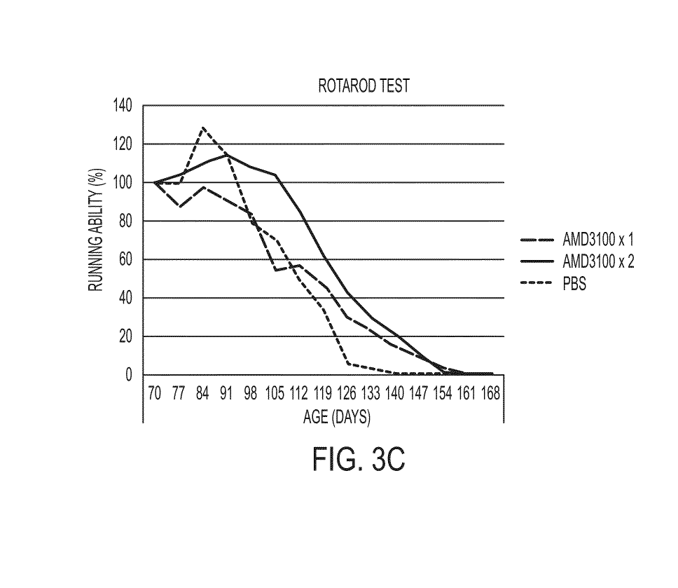

FIG. 3A shows mice that were treated with AMD3100 once and twice a week died at ˜170 days old, which is about 14 days more than control mice. The weight loss of these mice was also smaller than the weight loss of PBS mice (FIG. 3B). However, performance in the Rotarod test, which resembles motor function, was better among mice that received AMD3100 twice a week, whereas mice that received AMD3100 once a week performed similarly to the control group.

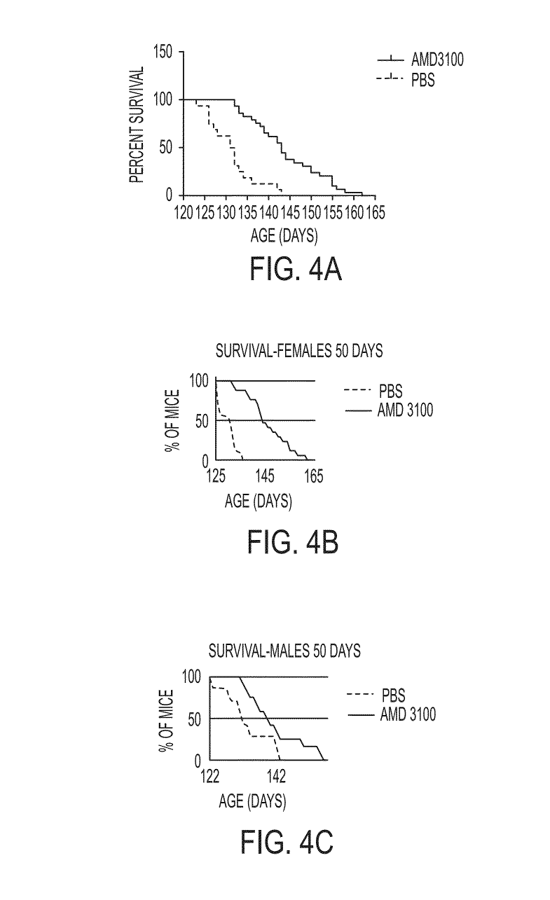

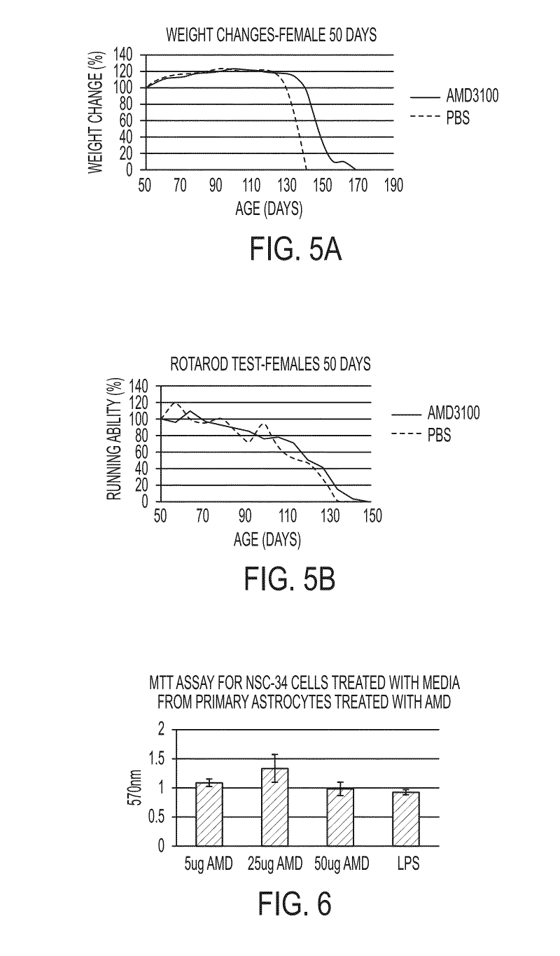

AMD3100 treatment for once and twice a week thus extended the survival in about 14 days, compared with mice treated with PBS (FIGS. 4A-4C). In addition, the treatment resulted in less weight loss as disease retrogrades (FIG. 5A). The improvement in motor function, detected by Rotarod test, was remarkable throughout disease progression mostly among mice treated twice a week with AMD3100, whereas mice treated only once a week showed improvement only at 50% of running ability or less.

All of the beneficial effects, together or individually, are significant to disease progression and the amelioration of ALS.

### Microglia Activation

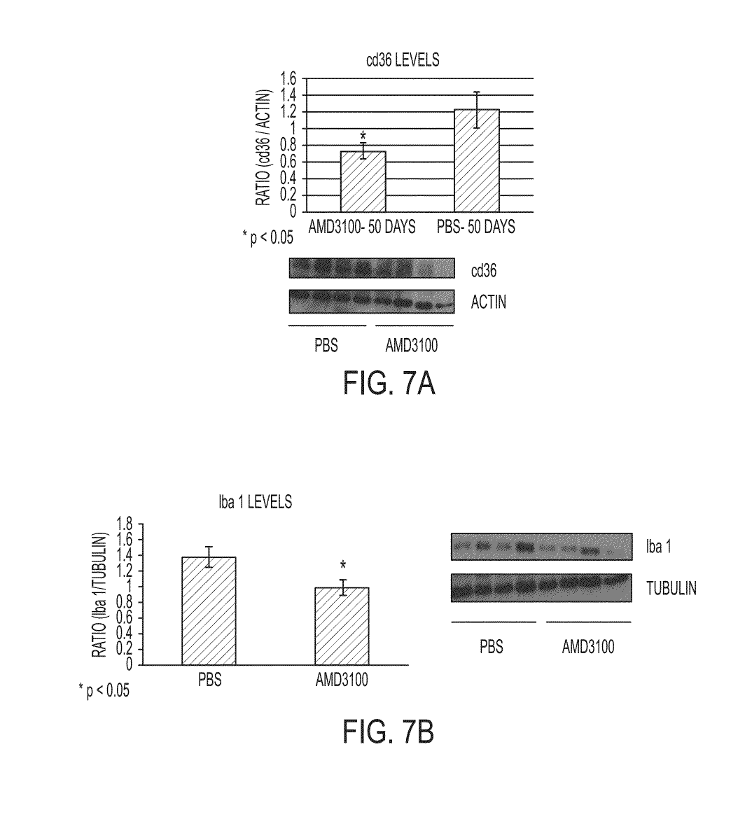

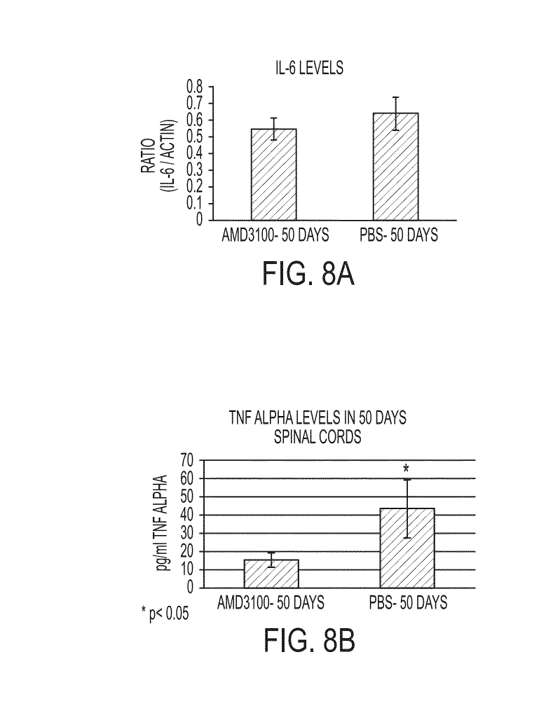

Loss of upper and lower MNs results in progressive muscle paralysis and ultimately death due to respiratory failure. Although initially thought to derive from the selective loss of MNs, the pathogenic concept of non-cell autonomous disease has come to the fore front for the contribution of glial cells in ALS, in particular microglia. Recent studies suggest that microglia may have a protective effect on MN in an early stage. Conversely, activated microglia contributes and enhances MN death by secreting neurotoxic factors, and impaired microglial function at the end-stage may instead accelerate disease progression. However, the nature of microglial-neuronal interactions that lead to MN degeneration remains elusive. The present inventors measured reduction of markers specific for activated microglia related to their contribution of the neurodegenerative network in ALS pathology, in the transgenic SOD1-G93A rodents, the most widely used model for the disease. All markers tested, including Iba-1 and cd36 resulted in significant reduction in its levels (FIG. 7A-7B).

### Restoration of BBB

In ALS patients and rodents expressing ALS-associated superoxide dismutase 1 (SOD1) mutations were reported alterations in the blood-Central Nervous System barrier (B-CNS-B) composed of the blood brain barrier (BBB), blood-spinal cord barrier (BSCB), and blood-cerebrospinal fluid barrier (BCSFB) as suggested from the reduction of levels of various tight junction proteins including zonula occludens-1 (ZO-1), occludin and claudin-5 between endothelial cells. The loss of tight junction proteins (TJPs) occludin and ZO-1 in the microvasculature has been also shown to be mediated by various cytokines such as monocyte chemoattractant protein-1 (MCP1), TNF-α, IL-1β, and IFN-γ. The reduction of the tight junction proteins resulted in microhemorrhage with release of neurotoxic hemoglobin-derived products, reductions in microcirculation and hypoperfusion.

SOD1 mutants are proposed to mediate endothelial damage even before motor neuron death and hypoxia and inflammation led to increased BSCB permeability and disruption. Early motor-neuron dysfunction and injury were shown to be proportional to the degree of BSCB disruption, and early protection of the BSCB integrity was found to delay onset of motor-neuron impairment and degeneration. Altogether, these findings in mice show that BSCB breakdown plays a role in early-stage disease pathogenesis and that restoring BSCB integrity retards the disease process.

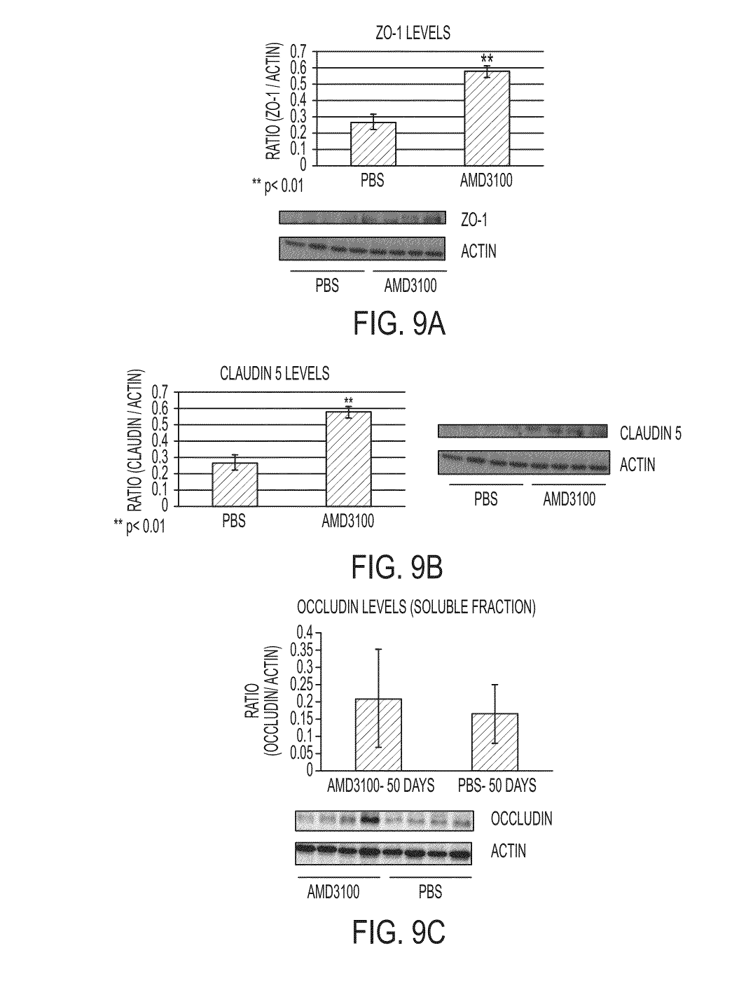

Endothelial cells play an important role in B-CNS-B function. B-CNS-B dysfunction is recognized to participate in neurodegenerative disorders. Cerebral vascular endothelial cells develop highly selective barrier which controls the exchanges between blood and brain or blood and spinal cord compartments for the maintenance and regulation of the neuronal microenvironment. TJPs such as ZO-1, occludin and the claudin family exist in cerebral vascular endothelial cells, which are the most crucial factors modulating barrier integrity. It has been suggested that B-CNS-B dysfunction in ALS and Alzheimer's disease (AD) are likely related to the injury or dysregulation of TJPs. The expression of TJPs has been reported to contribute to barrier function. ZO-1 and claudin-5 are the most important components for cell barrier integrity. Claudin-5 can greatly reduce dextran permeability and improve transendothelial electrical resistance and plays an essential role in the earliest stage of CNS angiogenesis. ZO-1 serves as a bridge between transmembrane proteins and skeleton proteins, and this interaction is important to the stability and function of endothelial barrier. Occludin has also been suggested to play a key role in the barrier function of the TJPs. All these three proteins are known to be reduced by 40%-60% in both animal models of ALS and humans, even prior to disease onset (Zhong et al., 2008). Therefore, the present inventors focused on these TJPs in an experimental study, and found that chronic administration of AMD3100 was effective in restoring the expression of these TJPs (FIGS. 9A-9C). This data reveal a novel pharmacological effect of AMD3100 on endothelial barrier, which improves the barriers and thus prevents additional damage.

### Neuronal Remyelinization

Oligodendrocytes in CNS, and Schwann cells in the peripheral nervous system (PNS), are responsible for the myelin sheaths surrounding neurons which provide electrical insulation essential for rapid signal conduction. Schwann cells also participate in the clearance of debris and in guiding the axon after neuron damage Schwann cells are in intimate contact with the full length of the axons of lower motor neurons. After axonal damage, Schwann cells also participate—in concert with peripheral macrophages—in clearing debris and in guiding the recovering axon (Ilieva et al., 2009).

In the spinal cord of ALS mice, there was found extensive degeneration of gray matter oligodendrocytes prior to disease onset. Although new oligodendrocytes were formed, they failed to mature, resulting in progressive demyelination. Oligodendrocyte dysfunction was also prevalent in human ALS, as gray matter demyelination and reactive changes in oligodendrocyte progenitors (NG2+ cells) were observed in motor cortex and spinal cord of ALS patients. Selective removal of mutant SOD1 from oligodendroglia substantially delayed disease onset and prolonged survival in ALS mice, suggesting that ALS-linked genes enhance the vulnerability of motor neurons and accelerate disease by directly impairing the function of oligodendrocytes (Kang et al., 2013). Moreover, selective excision of a dismutase-active mutant SOD1 from a substantial proportion of Schwann cells (70%) (through P0-Cre-mediated excision) yielded a highly unexpected outcome: not only did removal of the mutant gene fail to slow any aspect of disease, it generated a substantial acceleration of the late phase of disease (Lobsiger et al., 2009). In spinal cord injury, reactive gliosis emerges in the lesion accompanied by the up-regulation of chondroitin sulfate proteoglycans (CSPGs) in oligodendrocytes and Schwann cells. Although only a few studies have examined whether oligodendrocytes or myelin sheaths have a role in ALS, the myelin abnormalities consisting in loss of compact myelin and lamellae detachment in the spinal cord of pre-symptomatic SOD1 transgenic rats and aggravated at symptomatic stages (Lasiene and Yamanaka, 2011) suggest that it may be an interesting target for further study.

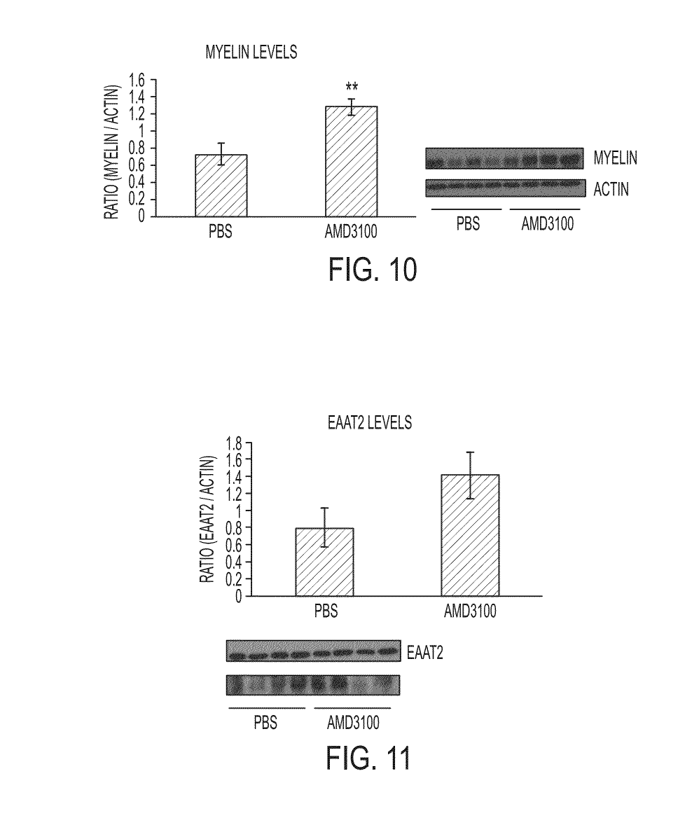

Chronic treatment with AMD3100 to SOD-G93A mice induced significant remyelinization in motor neurons in spinal cord of treated mice (FIG. 10), suggesting a novel therapeutic feature of AMD3100 for the first time. This ability of AMD3100 to induce remyalinization, might be effective in other neurodegenerative disease which involve pathological demyelinization.

### Glutamate Exitotoxicity

One of the early proposed mechanisms—observed both in SOD1 mutant mouse models and in familial and sporadic ALS patient samples—is glutamate excitotoxicity, the excessive firing of motor neurons derived from failure to rapidly remove synaptic glutamate. Overstimulation by glutamate, the neurotransmitter that triggers motor neurons to fire, can elicit a cascade of toxic events in the postsynaptic motor neuron including repetitive activation of glutamate receptors and the corresponding increase in calcium influx, thus overriding the storage abilities of mitochondria and endoplasmic reticulum (ER). Contributing to this phenomenon is a failure to rapidly clear extracellular glutamate through deficiency in the glutamate transporter EAAT2 in the astrocytic processes that surround synapses of motor neurons and is responsible for about 95% of total glutamate transport at the synapses (Le Verche et al., 2010).

In both familial and sporadic ALS cases there is evidence for loss of EAAT2 glutamate transporter, even before the onset of clinical symptoms (Howland et al., 2002). Studies of human sporadic ALS tissues showed loss of up to 95% of EAAT2 protein expression and activity in affected areas of the CNS (FIG. 11). This marked loss might be explained by truncated form of EAAT2, likely deriving from caspase-3-mediated proteolytic cleavage, which appeared concurrently to the loss of EAAT2 immunoreactivity and to increased expression of activated caspase-3, seen in spinal cord homogenates of mutant SOD1 ALS mice (Boston-Howes et al., 2006).

The significance of normal EAAT2 expression to neuronal survival was demonstrated by overexpressing the EAAT2 protein in mSOD1 mouse model astrocytes, which resulted in delay in disease onset, in addition to motor neuron survival. Similar outcomes were seen with drugs that increase EAAT2 expression (Guo et al., 2003).

The findings by the present inventors showed that chronic administration of AMD3100 to SOD-G93A mice resulted in increase in EAAT2 levels, which might result also in decrease in the exitotoxic levels of extracellular glutamate, another feature of AMD3100 that has been never described before.

Having now generally described the invention, the same will be more readily understood through reference to the following example which is provided by way of illustration and is not intended to be limiting of the present invention.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Allen, N. J. and Attwel,l D. 2001. A chemokine-glutamate connection. Nat Neurosci. 4(7):676-8.
Arimitsu, N., Shimizu, J., Fujiwara, N., Takai, K., Takada, E., Kono, T., Ueda, Y., Suzuki, T. and Suzuki, N. 2012. Role of SDF1/CXCR4 Interaction in Experimental Hemiplegic Models with Neural Cell Transplantation. Int J Mol Sci. 13(3):2636-49.
Azzouz et al., Nat. Med. 2005; 11(4):429-33
Boston-Howes, W., Gibb, S. L., Williams, E. O., Pasinelli, P., Brown, R. H. Jr. and Trotti, D. 2006. Caspase-3 cleaves and inactivates the glutamate transporter EAAT2. J Biol Chem. 19; 281(20):14076-84.
Esté et al., Activity of different bicyclam derivatives against human immunodeficiency virus depends on their interaction with the CXCR4 chemokine receptor. Molecular Pharmacology 55:67-73 (1999).
Faber et al., The many facets of SDF-1α, CXCR4 agonists and antagonists on hematopoietic progenitor cells. J Biomed Biotechnol 2007(3):26065 (Epub Apr. 23, 2007).
Gerlach et al., Metal ion enhanced binding of AMD3100 to Asp262 in the CXCR4 receptor. Biochemistry 42:710-717 (2003).
Guo, H., Lai, L., Butchbach, M. E., Stockinger, M. P., Shan, X., Bishop, G. A., Lin, C. L. 2003. Increased expression of the glial glutamate transporter EAAT2 modulates excitotoxicity and delays the onset but not the outcome of ALS in mice. Hum Mol Genet 12:2519-2532.
Henkel J S, Beers D R, Zhao W, Appel S H (2009) Microglia in ALS: the good, the bad, and the resting. J Neuroimmune Pharmacol 4:389-398.
Howland, D. S., Liu, J., She, Y., Goad, B., Maragakis, N. J., Kim, B., Erickson, J., Kulik, J., DeVito, L., Psaltis, G. 2002. Focal loss of the glutamate transporter EAAT2 in a transgenic rat model of SOD1 mutant-mediated amyotrophic lateral sclerosis (ALS). Proc Natl Acad Sci USA 99:1604-1609.
Ignacio et al., Ann. N.Y. Acad. Sci. 2005, 1053:121-136.
Ilieva. H., Polymenidou. M., Cleveland. D. W. (2009) Non-cell autonomous toxicity in neurodegenerative disorders: ALS and beyond. J Cell Biol. 14; 187(6):761-72.
Kang S H, Li Y, Fukaya M, Lorenzini I, Cleveland D W, Ostrow L W, Rothstein J D, Bergles D E. (2013) Degeneration and impaired regeneration of gray matter oligodendrocytes in amyotrop hic lateral sclerosis. Nat Neurosci. 16(5):571-9.
Kieran et al., Nat Med 2004; 10(4):402.
Kieran and Greensmith, Neurosci 125(2):427-39 (2004).
Lasiene J, Yamanaka K. (2011) Glial cells in amyotrophic lateral sclerosis. Neurol Res Int. 2011:718987.
Le Verche, V., Ikiz, B., Jacquier, A., Przedborski, S., and Re, D. B. 2010. Glutamate pathway implication in amyotrophic lateral sclerosis: what is the signal in the noise? J Receptor Ligand Channel Res. 2011:4:1-22.
Lobsiger, C. S., Boillee, S., McAlonis-Downes. M., Khan, A. M., Feltri, M. L., Yamanaka, K., Cleveland, D. W. (2009) Schwann cells expressing dismutase active mutant SOD1 unexpectedly slow disease progression in ALS mice. Proc. Natl. Acad. Sci. USA. 106:4465-4470.
Petit, I., Jin, D. and Rafii, S. 2007. The SDF-1-CXCR4 signaling pathway: a molecular hub modulating neo-angiogenesis. Trends Immunol. 28(7):299-307
Pusic, I. and DiPersio, J. F. 2010. Update on clinical experience with AMD3100, an SDF-1/CXCL12-CXCR4 inhibitor, in mobilization of hematopoietic stem and progenitor cells. Curr Opin Hematol. 17(4):319-26.
Reinholz et al., Exp Neurol. 1999; 159(1):204-16.
Sathasivam et al., 2005 Neuropath App Neurobiol. 31(5):467
Vargas, M. R., Johnson, J. A. 2010. Astrogliosis in amyotrophic lateral sclerosis: role and therapeutic potential of astrocytes. Neurotherapeutics. 7(4):471-81.
Zhong. Z., Deane. R., Ali. Z., Parisi. M., Shapovalov. Y., O'Banion. M. K., Stojanovic. K., Sagare. A., Boillee. S., Cleveland. D. W., Zlokovic. B. V. (2008) ALS-causing SOD1 mutants generate vascular changes prior to motor neuron degeneration. Nat Neurosci. 11 (4):420-2.

1-3. (canceled)

4. A method for treating amyotrophic lateral sclerosis (ALS), comprising administering to a patient suffering from ALS an effective amount of a bicyclam inhibitor of G protein-coupled receptor CXCR4.

5. The method of claim 4, wherein the inhibitor of CXCR4 is 1,1′-[1,4-Phenylenebis(methylene)]bis-1,4,8,11-tetraazacyclotetradecane or a pharmaceutically acceptable salt thereof.

6. The method of claim 4, wherein said inhibitor is provided in an amount effective for inhibiting glutamate release in astrocytes.

7. (canceled)

8. The method of claim 4, wherein said inhibitor is provided in an amount effective for increasing remyelinization in motor neurons.

9. (canceled)

10. The method of claim 4, wherein said inhibitor is administered once a week.

11. The method of claim 4, wherein said inhibitor is administered twice a week.
